SEQUENCE LISTING (7 sequences)

SEQ ID NO: 1 - DNA, 19 bp, Artificial Sequence
  Oligonucleotide Primer
ttttttttttttttttttu

SEQ ID NO: 2 - DNA, 19 bp, Artificial Sequence
  Oligonucleotide Primer
aaaaaaaaaaaaaaaaaau

SEQ ID NO: 3 - DNA, 18 bp, Artificial Sequence
  Oligonucleotide Primer
gaccggcgctcagctgga

SEQ ID NO: 4 - DNA, 18 bp, Artificial Sequence
  Oligonucleotide Primer
gcctctgttccgcagatt

SEQ ID NO: 5 - DNA, 20 bp, Artificial Sequence
  Oligonucleotide Primer
cttgaacagtctgattaaac

SEQ ID NO: 6 - DNA, 16 bp, Artificial Sequence
  Oligonucleotide Primer
gctggggctgcctgcc

SEQ ID NO: 7 - DNA, 16 bp, Artificial Sequence
  Oligonucleotide Primer
ggccgcctgacgccga

(19) United States
(12) Patent Application Publication
(10) Pub. No.: US 2002/0031776 A1
(43) Pub. Date: Mar. 14, 2002

(54) Enzymatic labeling and detection of DNA hybridization probes

(76) Inventors: Richard H. Tullis, Encinadas, CA (US); Jerome A. Streifel, San Diego, CA (US)

Correspondence Address: Stephanie Seidman, Heller Ehrman White & McAuliffe LLP, 6th Floor, 4350 La Jolla Village Drive, San Diego, CA, 92122, US

(21) Appl. No.: 09/917,138

(22) Filed: Jul. 26, 2001

Related U.S. Application Data
(63) Continuation of application No. 09/580,358, filed on May 25, 2000.
(60) Provisional application No. 60/136,545, filed on May 28, 1999.

(51) Int. Cl. C12Q 1/68; C12P 19/34

(52) U.S. Cl. 435/6; 435/91.2

(57) ABSTRACT

Methods and markers for identifying and detecting nucleic acids using a linear amplification scheme are provided. The methods use chain extending enzymes, such as telomerases, to label probes hybridized to target nucleic acid products, including amplification products, to render them readily detectable. The methods may be performed as homogeneous or heterogeneous reactions. The methods provided herein can be used with any known method involving detection of a hybridized probe.

### RELATED APPLICATIONS

This application is a continuation of U.S. application Ser. No. 09/580,358, filed May 25, 2000, to Richard H. Tullis and Jerome A. Steifel, entitled “ENZYMATIC LABELING AND DETECTION OF DNA HYBRIDIZATION PROBES.” Benefit of priority under 35 U.S.C. §119(e) to U.S. provisional application Ser. No. 60/136,545, filed May 28, 1999, to Richard H. Tullis and Jerome A. Steifel, entitled “ENZYMATIC LABELING AND DETECTION OF DNA HYBRIDIZATION PROBES” is claimed herein. The contents and subject matter of the provisional application and U.S. application Ser. No. 09/580,358 are herein incorporated by reference in their entirety.

### FIELD OF THE INVENTION

Methods and markers for identifying and detecting nucleic acids using a linear amplification scheme are provided. More particularly, the methods employ chain extending enzymes to label amplification products permitting easy detection.

### BACKGROUND OF THE INVENTION

DNA probes and primers have found a variety of commercial and research application in DNA hybridization diagnostics including DNA and RNA target amplification technologies (PCR, LCR and NASBA); signal amplification technologies such as branched DNA probes, dendrimers and the like; and direct DNA probes for less sensitive detection.

The concept of DNA hybridization was first worked out in the late 1950's and early 1960's in studies on the melting behavior of purified viral DNA and homopolymer tracts (P. Doty (1962) Biochem. Soc. Symposia 21:8). The application of such techniques was quickly recognized as an important diagnostic tool (see, e.g., U.S. Pat. No. 4,358,535).

Up until 1983, most DNA detection methods were based upon radioautography. Radioautographic detection limits using DNA hybridization probes for viruses and small DNA plasmids average 1-5 picograms (˜10⁶ molecules) of target DNA in an overnight exposure (Brandsma et al. (1982) Proc. Natl. Acad. Sci. U.S.A. 77:6851; and Kafatos et al. (1979) Nucl. Acids Res. 7:1541). Since illness can be caused by a single virus, higher sensitivity assays were still needed.

The need for more sensitive and stable DNA hybrid detection systems was altered when the first exponential DNA amplification procedure, polymerase chain reaction (PCR) was developed (see, U.S. Pat. No. 4,683,195). PCR uses a thermocycling system together with a heat stable polymerase to amplify a target nucleic acid over 1 trillion-fold allowing single molecules of DNA or RNA to be detected.

Since the advent of PCR, a number of viable methods have been developed that are in use for various applications. In general, amplification strategies can be divided between signal amplification and target amplification. Examples of signal amplification include Branched DNA (B-DNA) (U.S. Pat. No. 5,124,246) and cycling probe amplification (U.S. Pat. No. 5,660,998). Examples of target amplification include linear amplification (U.S. Pat. No. 5,837,450), NASBA (U.S. Pat. No. 5,409,818), 3SR (U.S. Pat. No. 5,399,491), Strand Displacement Amplification (U.S. Pat. No. 5,455,166), Ligase Chain Reaction (LCR-Abbott, Chicago, Ill.) and Polymerase Chain Reaction (U.S. Pat. Nos. 4,683,202 and 4,683,195).

While amplification products may be visualized using gel electrophoresis, the most widely used method for discriminating among amplified sequences, particularly to distinguish and identify polymorphisms, is based on nucleic acid hybridization. Typically, an oligonucleotide probe labeled with a detectable reporter group, (e.g., ³²p, biotin, digoxigenin, alkaline phosphatase or horseradish peroxidase (HRP)) is made for each known sequence polymorph. The oligonucleotide is then hybridized to an immobilized amplicon and detected using the appropriate instrumentation. Alternatively, the oligonucleotide probe may be immobilized on a solid support (e.g., a nylon membrane or plastic microtiter plate) and hybridized to an amplicon containing a reporter group incorporated using amplification. While all of these techniques have been applied and are in use in various laboratories, the most convenient and cost effective employ immobilized sequence specific oligonucleotides.

This type technology has most recently been implemented in miniaturized hybridization arrays or gene chips (see, e.g., Gress et al. (1992) Mamm. Gemone 3:609-619; Shalon et al. (1996) Genome Res. 6:639-45; Schena et al. (1995) Science 270:467-470; Pietu et al. (1996) Genome Res. 6:429-503). Gene chips using a photochemical DNA synthesis technique to synthesize microarrays containing up to 10⁵ specific oligonucleotides on a single silicon chip have been developed (Affymax, Palo Alto, Calif.).

Similar technology employing electronically active gene chips has been introduced by Nanogen (San Diego, Calif.; Sosnowski et al (1997) Proc. Natl. Acad. Sci., USA 94:1119-23). In these systems, each array element is individually electronically addressed permitting stringent hybridization for detecting single base mismatches without adjusting bulk solvent or temperature conditions.

Another system that has been applied to genotyping is the Taqman system (Perkin Elmer, Foster City, Calif.). In the Taqman paradigm (see, e.g., Holland et al. (1991) Proc. Natl. Acad. Sci. U.S.A. 88:7276-7280), fluorescent energy-transfer probes known as Taqman probes or Molecular Beacons have been employed in a homogeneous format to detect amplification products. A Taqman probe includes a fluorescent donor and fluorescent quencher typically attached to the 3′ and 5′ ends of a sequence specific oligonucleotie (SSO). In a Molecular Beacon, the quencher is a non-fluorescent chromophore, such as, but are not limited to, DABCYL (4-(4-dimethylaminophenyl)azobenzoic acid; see, e.g., Kostrikis et al. (1996) Science 279:1228-1229) and EDANS (5-((2-amino-ethyl)amino)-naphthalene-1-sulfonic acid), which is fluorescent group quenched by the DABCYL group. During amplification, the exonuclease activity of Taq polymerase cleaves the probe between the quencher and the fluor, causing a directly observably increase in fluorescence of from 3-20 fold. The Taqman system combines the amplification and detection in a closed system reducing the risk of contamination and allowing multiplex detection. There are drawbacks to this system. Taqman probes vary substantially in quenching efficiency and are difficult to synthesize and purify. As a result, the system tends to be less robust than typical clinical systems and cannot use highly modified DNA probes that are resistant to nucleases. Moreover, Taqman probes and the associated instrumentation to detect fluorescence changes can be quite expensive.

Coupling amplification with a sensitive non-isotopic detection technique developed for direct DNA hybridization diagnostics has reduced the overall assay time and improved quantitation. Non-radioactive detection systems using colorimetry are widely employed to detect amplification products. These protocols typically involve the use of indirect recognition labels (for immobilization) and direct reporter labels (for detection). Indirect recognition labels include biotin and digoxigenin as well as structural features of the amplified product that can be recognized immunochemically or through the use of DNA binding proteins. Reporter labels are used indirectly in the form of conjugates or fusion proteins as well as through direct attachment to probes or primers. Direct reporter labels include enzymes (e.g., alkaline phosphatase), fluorophores and chemiluminescent molecules (e.g., acridinium esters and isoluminol derivatives).

Advances in the application of these techniques in combination with sensitive fluorescent labels have led to methods that allow the detection of single DNA molecules. With the advent of PCR and in situ PCR, single molecules can be detected but not localized. It is possible to visualize single DNA probes (Castro et a. (1997) Anal, Chem. 69:3915-3920) as well as long DNA molecules synthesized by the rolling circle technique (Lizardi et al. (1998) Nature Genetics 19:225-232; Lockey et al. (1998) Biotechniques 24:744-6; U.S. Pat. No. 5,714,320). For those techniques in which amplification is used, fine structure localization is limited.

Notwithstanding these approaches to nucleic acid amplification and detection needs available, improvements in DNA probe technologies continue to provide new structures exemplified by DNA chips and nanotechnology applications that are useful in diagnostics. Sensitive detection of hybridization events, however, remains a principal focus in DNA diagnostics. The reason for this interest is that relatively simple amplification technologies are sensitive but are difficult to quantify, while direct hybridization detection is quantitative but relatively insensitive.

Thus, new highly sensitive techniques that simplify direct detection are needed. Therefore, it is an object herein to provide methods that permit highly sensitive direct detection.

### SUMMARY OF THE INVENTION

The methods provided herein rely on the use of chain extending enzymes, such as terminal deoxynucleotidyl-transferase (terminal transferase) [EC 2.7.7.31], to label amplification products. Terminal transferase has long been used to tail DNA fragments for cloning and attachment to membranes (see, e.g., Hofstetter et al. (1976) Biochem, Biophys. Acta 454:587 and Maniatis et al. (1982) in Molecular Cloning: A Laboratory Manual, Cold Spring Harbor Lab, Cold Spring Harbor, New York (1982)).

Terminal transferase has been used to label DNA probes. In situ applications have proven impossible since terminal transferase non-specifically labels all available DNA termini and most often will label it with truncated chain extension products. The methods provided herein solve this problem.

In particular, the method provides a way to prevent non-specific chain growth while allowing extended chain growth from hybridized DNA probes, thereby affording highly sensitive detection of DNA hybridization events is provided.

In embodiments of the method provided herein, a sample containing the nucleic acid to be detected is prepared for reaction with a nucleic acid hybridization probe. The sample may be either in solution or attached to a solid phase, such as, but are not limited to, a glass slide and silicon support. The sample is then treated with a capping reagent, typically a dideoxynucleoside triphosphate, to block any naturally occurring substrates for the chain elongation reaction. The capping reagent is then removed and the sample is hybridized with a nucleic acid probe specific for a desired target. When hybridization is complete, unbound probe is removed. Probe molecules bound to the sample are then labeled in situ using a chain extending polymerase such as terminal transferase and a nucleoside triphosphate labeled with a suitable reporter group (e.g. fluorescein) to produce a long tail on the probe which can easily be detected. Unincorporated triphosphates are then removed and the remaining reporter groups in the newly formed tails detected using a suitable detection system.

### DETAILED DESCRIPTION OF THE INVENTION

A. DEFINITIONS

Unless defined otherwise, all technical and scientific terms used herein have the same meaning as is commonly understood by one of skill in the art to which this invention belongs. All patents, applications, published applications and other publications and sequences from GenBank and other data bases referred to anywhere in the disclosure herein are incorporated by reference in their entirety.

As used herein, a non-template dependent chain extending enzyme refers to template independent polymerases capable of adding polynucleotide tails to the termini of DNA or RNA molecules. Chain expending enzymes include, but are not limited to, telomerases such as terminal transferases, that are capable of producing extended polynucleotide tails. Telomerases extend the 3′ termini of chromosomes thereby stabilizing chromosomal structure. Assays to identify telomerases are known (see, e.g., U.S. Pat. Nos. 5,489,508, 5,645,986 and 5,648,215). Generally telomerase activity is measured by primer chain elongation under conditions that minimize interference from other genomic sequences. For example, U.S. Pat. No. 5,629,154 describes telomerase activity assays. In these assays, telomerase activity in a sample is measured using a two reaction protocol involving telomerase substrate and primer extension steps.

As used herein, nucleic acid probes or DNA probes are polymers of nucleobases covalently coupled into an extended chain and capable of specifically pairing with nucleic acids found in viruses, bacteria and all higher organisms. The linkages that connect the nucleobases can include non-ionic or ionic moieties, for example, modified bases and protein nucleic acid (PNA). Probes are typically at least about 8 nucleotides, typically at least 10 nucleotides and generally more than about 14-16 nucleotides, and substantially longer. Probes are typically designed to specifically bind to a target sequence in a nucleic acid molecule under selected conditions of hybridization stringency.

As used herein, a nucleic acid molecule refers to DNA or RNA. A modified nucleic acid molecule refers to nucleic acid molecules that include one or more modified nucleotides incorporated into the chain, and includes nucleic acid molecules that have altered linkages. Modified bases, include, but are not limited to, bases with mass modifications or substitutions on the sugar or phosphate backbone, in the base or include nucleotides other than G, C, T(U) or A.

As used herein, reporter groups refer to any chemical moiety that renders a hybridization reaction detectable. Examples of reporter groups include, but are not limited to, radiolabels, such ³²P, chemical labels, biotin, digoxigenin, fluorescent labels, such fluorescein and enzyme labels, such as horse radish peroxidase and a luciferase.

As used herein, stringency refers to the conditions for performing hybridization reactions, particularly the conditions selected for the final wash, and selected conditions depend upon the desired specificity for which hybridization is performed. For use with the methods herein, any stringency may be used. For example, high stringency is achieved using a combination of high temperature for hybridization and low salt concentration for washing based initially on theoretical calculations. The equations used in calculations of the melting temperature (Tm) and reaction rates are well known (see, e.g., Britten, et al., Methods in Enzymol, 29E:363 (1974); J. Meinkoth, et al., Anal. Biochem, 138:267-84 (1984); J. G. Wetmur, et al., J. Mol. Biol., 31:349 (1967) and R. Weidner, et al., biopolymers, 20:1537-47 (1981)). In general, stringencies are most easily varied using fixed hybridization and washing temperatures under changing monovalent cation concentrations. Exemplary stringencies for washing hybrids are as follows:

1) high stringency: 0.1× SSPE (or SSC), 0.1% SDS, 65° C.

2) medium stringency: 0.2× SSPE (or SSC), 0.1% SDS, 50° C.

3) low stringency: 1.0× SSPE (or SSC), 0.1% SDS, 50° C.

It is understood that equivalent stringencies may be achieved using alternative buffers, salts and temperatures. The recipes for SSC, SSPE and Denhardt's and the preparation of deionized formamide are described, for example, in Sambrook et al. (1989) Molecular Cloning, A Laboratory Manual, Cold Spring Harbor Laboratory Press, Chapter 8.

B. Practice of the Method

Methods for rendering probes hybridized to nucleic acid molecules in a sample thereof are provided. The methods rely on non-template dependent extension of the hybridized probes. Prior to hybridization the nucleic acid molecules in a sample are treated to render them non-extendible by a non-template dependent chain extending enzyme. These methods are intended for use in any method that includes detection of hybridized probes.

The methods provided herein permit highly sensitive detection using oligonucleotide probes, including non-ionic and highly modified oligonucleotide probes (e.g. PNAs, methylphosphonates or morpholino oligonucleotide probes). Furthermore, since non-ionic DNA probes and probes including modified bases, typically are not substrates for polymerase reactions they generally cannot be used as primers for amplification reactions and detection thereof. The methods herein, provide a means to use such probes for amplification and detection. The only requirement for use of probes in the method herein, is the inclusion or addition of a short nucleic acid segment, of at least about 3 bases, to any modified probe. This provides a priming site for template independent chain elongation. Thus the present method allows these exotic DNA probes to be amplified and readily detected.

Hence, the methods provided herein provide means for labeling the product of a variety of amplification reaction products, and can be adapted for use with virtually any method that involves detection of a labeled oligonucleotide probe. The U.S. patents, set forth in the following table, describe exemplary amplification reactions that can incorporate the labeling method provided herein to improve sensitivity of the disclosed methods:

TABLE 1

| First | | | |
|---|---|---|---|
| named | Year | | U.S. Pat. |
| inventor | issued | Title | No. |
| Mullis | 1987 | Process for amplifying, detecting, | 4,683,195 |
| | | and/or cloning nucleic acid sequences | |
| Mullis | 1987 | Process for amplifying nucleic acid | 4,683,202 |
| | | sequences | |
| Mullis | 1989 | Process for amplifying, detecting, | 4,800,159 |
| | | and/or cloning nucleic acid sequences | |
| Davey | 1988 | Nucleic acid amplification process | 5,409,818 |
| Urdea | 1988 | Polynucleotide determination with | 4,775,619 |
| | | selectable cleavage sites | |
| Kramer | 1988 | Autocatalytic replication of recombinant | 4,786,600 |
| | | RNA | |
| Chu | 1990 | Replicative RNA reporter systems | 4,957,858 |
| Kacian | 1992 | Nucleic acid sequence amplification | 5,399,491 |
| | | methods | |
| Malek | 1992 | Enhanced nucleic acid amplification | 5,130,238 |
| | | process | |
| Dattagu | 1993 | Nucleic acid amplification employing | 5,215,899 |
| pta | | ligatable hairpin probe and transcription | |
| Axelrod | 1994 | Replicative RNA-based | 5,356,774 |
| | | amplification/detection system | |
| Jones | 1995 | Method for retrieval of unknown | 5,411,875 |
| | | flanking DNA sequence | |
| Dahlberg | 1995 | Method of site specific nucleic acid | 5,422,253 |
| | | cleavage | |
| Kramer | 1996 | Method of using replicatable | 5,503,979 |
| | | hybridizable recombinant RNA probes | |
| Munroe | 1997 | Interspersed repetitive element-bubble | 5,597,694 |
| | | amplification of nucleic acids | |
| Tyagi | 1998 | Sensitive nucleic acid sandwich | 5,759,733 |
| | | hybridization assay | |
| Kool | 1998 | Rolling circle synthesis of | 5,714,320 |
| | | oligonucleotides and amplification of | |
| | | select randomized circular | |
| | | oligonucleotides | |
| Dahlberg | 1998 | Detection of target nucleic acid | 5,837,450 |
| | | molecules using thermostable 5′ | |
| | | nuclease | |
| West | 1996 | Therapy and diagnosis of conditions | 5,489,508 |
| | | related to telomere length and/or | |
| | | telomerase activity | |
| Kim | 1997 | Telomerase activity assays | 5,629,154 |
| West | 1997 | Therapy and diagnosis of conditions | 5,645,986 |
| | | related to telomere length and/or | |
| | | telomerase activity | |
| West | 1997 | Telomerase diagnostic methods | 5,648,215 |

Practice of an Exemplary Embodiment

In a preferred embodiment, the method provided herein includes the following steps:

First, all of the unwanted reactive sites in the sample containing or potentially containing the target nucleic acid molecule or particular sequence of nucleic acids in a molecule, are capped otherwise rendered inaccessible for further chain extension. This can be achieved, for example, with terminal transferase and a dideoxynucleoside triphosphate (ddNTP) or other chain terminating reagent.

Second, the sample containing the target is exposed to or reacted with a polynucleotide or modified nucleic acid probe, such as protein nucleic acid (PNA), under conditions such that hybrids of the desired complementarity can form. Then, unbound probe is washed away under conditions of selected stringency, such as, for example, highly stringent conditions that do not tolerate substantial mismatch whereby only probe bound to the desired target remains.

Third, the probe is extended in situ using a chain extending enzyme, such as terminal transferase and labeled nucleoside triphosphates, preferably to a chain length greater than about 100 bases, more preferably greater than about 400 bases, and most preferably greater than about 1000 bases, thereby providing a means for highly sensitive detection.

Step 1: Sample Preparation and Capping

A sample, which includes samples can be from any clinical, biological or environmental source, is obtained. For biological samples such as tissue sections or cultured cells, the materials may be usefully prepared by attachment to glass slides suitable for microscopic examination or by attachment to any solid support. Alternatively, nucleic acids may be isolated from samples such as blood or serum using standard techniques such as phenol:chloroform extraction and alcohol precipitation. Sample sources and methods for preparing samples for performing hybridization reactions are well known to those of skill in the art.

After sample preparation is complete, the sample is treated to render the nucleic acid molecules in the sample unsuitable for subsequent extension with the telomerase and other terminal transferase enzymes used in the extending step. Accordingly, the sample is treated to cap any 3′ ends to prevent them from serving as a substrate for the chain extension/labeling reaction.

Capping of oligonucleotide primers is well known in the art. For example, in the chemical synthesis of DNA oligonucleotides, unreacted change growth sites are capped using acetic anhydride. In DNA sequencing, chain growth is terminated by the addition of limiting amounts of dideoxynucleotides. Similarly, many nucleoside antibiotics such as AZT work by terminating DNA replication catalyzed by the incorporation of a non-extendable nucleoside. Hence a chain extending enzyme, such as a telomerase, is added in combination with chain terminators. Chain growth initiated by terminal transferase can be terminated by, for example, addition of any of 3-amino dNTPs, dideoxy-NTPs, ribo-NTPs (which typically add 2-3 nt before terminating) and any other known chain terminators.

Step 2: Probe Hybridization and Washing

After the capping reaction has been carried out, the sample is reacted with probe under the selected hybridization conditions. Typical hybridization conditions include contacting the sample with solution containing the DNA probe dissolved in a high salt buffer (e.g. 1 M sodium phosphate, pH 7.4) and incubating the mixture at elevated temperature for several hours. In this method, the composition of the hybridization and wash buffers and the temperature at which they are used are the primary determinants of accurate hybridization (stringency). Since every probe may have different melting temperatures and the purposes for which hybridization is conducted vary, conditions for the reaction, such as conditions for optimum specificity, may be slightly different. Hybridization conditions can be readily empirically determined.

Following hybridization, unbound probe is washed away under typically stringent conditions. Selected conditions for washing will depend upon the purpose for which hybrids were produced. For example, for samples with nucleic acids fixed to a solid surface, the immobilized material is washed with a low salt buffer (e.g. 1× SSC, 0.1% SDS). For samples hybridized in solution, unbound probe, may be removed, for example, by a variety of methods including enzymatic digestion with nucleases (e.g. S1 nuclease) or gel exclusion chromatography.

Step 3: Labeling and Detection

In a preferred embodiment, the chain extending enzyme is terminal transferase. Terminal transferase catalyzes a template independent addition of deoxynucleotides to the 3′ hydroxy termini of single or double stranded DNA molecules with the release of inorganic pyrophosphate (see, e.g., Bollum (1974) in The Enzymes, Vol. 19, P D Boyer (ed.) Academic Press, NY; Deng et al. (1983) Methods in Enzymol 100:96). Terminal transferase can efficiently incorporate biotinylated and fluorescent nucleotides (see, Vincent (1982) Nucl. Acids Res. 10:6787) and also will accept dideoxy- and ribonucleotide triphosphates under proper ionic conditions. Nucleotides and nucleoside analogs with modifications the 2′ and/or 3′ positions generally are acceptable substrates for terminal transferase and are incorporated into the extended chain (see, e.g., Hinton et al. Nucl. Acids Res. 10:1877-1894).

Terminal transferase requires only a short segment of DNA to prime synthesis of long chains. Thus, attachment of short DNA segment greater than or equal to 3 bases in length, is sufficient to allow chain growth even on highly modified or non-ionic DNA probes which cannot otherwise be used in amplification/detection reactions. Hence the probes for use in the methods herein should be so-designed.

This step provides for improved detection, since longer chains contain more label and are thus more easily detected. While this has been appreciated in the art, it has not generally proved possible to make long chain tails >1,000 nucleotides using terminal transferase. The methods provided herein, provide means to routinely produce nucleic acid polymers, particularly, DNA polymers, greater than 10 kb in length.

The products of the chain extension reaction can then be detected by suitable methods known to those of skill in the art. Suche methods include, but are not limited to:

1) Direct luminescent detection via incorporated fluorescence or chemiluminescent nucleoside triphosphates.

2) Indirect fluorescence or chemiluminescence mediated by antibodies, streptavidin or other lectins or aptamers

3) Enzymatic reporter groups attached to antibodies, streptavidin or other lectins or aptamers

4) Up convering phosphors or fluorescent beads attached to oligomers.

Hence, suitable labels include any detectable label that can be incorporated into an extended chain.

The following examples are included for illustrative purposes only and are not intended to limit the scope of the invention.

### EXAMPLE 1

### Preparation of DNA Oligonucleotide Probes

Synthesis of oligonucleotide probes was performed using standard phosphoramidite chemistry essentially as described by Beaucage and Caruthers (1984). In brief, fully blocked and carefully dried nucleoside phosphoramidites dissolved in anhydrous acetonitrile were sequentially added to the 3′ hydroxy terminal nucleotide bound to controlled pore glass supports via a succinate spacer (Matteucci and Caruthers, 1980). Nucleoside addition was followed by capping of unreacted 5′ hydroxyis with acetic anhydride, iodine oxidation, and 5′ detritylation in 2.5% trichloroacetic acid-methylene chloride. The resin bound oligomers were then dried by extensive washing in anhydrous acetonitrile and the process repeated. Condensation efficiencies of >98% were typically achieved as judged by trityl release. At the end of the synthesis, the finished resin was deblocked by brief treatment with concentrated ammonium hydroxide at 55° C. to remove the probe from the column and release the base blocking groups. The oligomers were then purified by HPLC (Oligo R3 columns eluting 0-60% acetonitrile containing 50 mM TEAA, pH 7.6), desalted, dried and stored for use.

Probes which have been synthesized and used in tailing reactions to produce tailed primers >1000 bases long are given in Table 2.

TABLE 2
Exemplary Primers Tailed to >1000 nt

| DNA Primer | Sequence | SEQ ID |
|---|---|---|
| Biotin-dT18U | Biotin- TTT TTT TTT TTT TTT TTT U | 1 |
| dA18U | AAA AAA AAA AAA AAA AAA U | 2 |
| Lambda RC | Biotin - GA CCG GCG CTC AGC TGG A | 3 |
| Biotin^{↑} | | |
| Lambda RC | Biotin - GA CCG GCG CTC AGC TGG A | 3 |
| Biotin^{↑↑} | | |
| DQA 5502c | GC CTC TGT TCC GCA GATT | 4 |
| DQA 7504c | CTT GAA CAG TCT GAT TAA AC | 5 |
| DQB 5705 | G CTG GGG CTG CCT GCC | 6 |
| DQB 5706 | GG CCG CCT GAC GCC GA | 7 |
| DQB 5707 | GG CCG CCT GCC GCC GA | 7 |

↑TTP: fluorescein-dUTP (10:1)
↑↑TTP: biotin-dUTP (10:1)

### EXAMPLE 2

Tailing Reactions Producing Long Polynucleotide Tails

Materials

Terminal transferase was obtained from commercial sources (Molecular Biology Resources (Milwaukee, Wis.) and Roche Boehringer-Mannheim, Germany). Reaction conditions for producing an acceptably long tailed DNA probe in a 50 ul volume containing 0.01 to 0.1 nmole of an oligonucleotide probe are 1000 uM dNTP, 100 mM sodium cacodylate buffer, pH 7.2, 0.2 mM mercaptoethanol, 50 ug/ml yeast inorganic pyrophosphatase and 2 mM CoC₂. The reactions can also be performed at room temperature or carried out at 37° C.

Results

Substantial incorporation, as evidenced by high performance gel filtration chromatographic (HPGFC) analysis of reaction products, incorporation of TTP and/or 5-fluorescein dUTP into primers was observed. The kinetics of incorporation was measured. The rate of incorporation of TTP over an 18 hour reaction at 37° C. using various primers and concentrations. Under the conditions of these reactions, incorporation was linear as a function of time for up to 4 hours; and incorporation continued for as long as 20 hours. Tail length was limited by the available dNTPs and primer concentration. Table 3 lists exemplary primers extended and the percent of TTP incorporated after 4 hours (sequences of the probe are set forth in Table 2).

TABLE 3

| | | Approximate % TTP |
|---|---|---|
| | | Incorporated after |
| Primer | concentration | about 4 hours |
| (AGCT)₅U | 1 nmol | ˜10% |
| BIOTIN-dT18U | 1 nmol | ˜20% |
| BIOTIN-dT18U | 2 nmol | ˜20% |
| BIOTIN-dT18U | 4 nmol | ˜20% |
| lambda capture | 0.5 nmol | ˜35% |
| DQA7504c | 1 nmol | ˜65% |

In a typical labeling reaction, there was a 10:1 molar ratio of unlabeled to labeled nucleotide (i.e. fluorescein dUTP and TTP). The longest tailed DNA probes were achieved by reducing the amount of primer relative to the input concentration of deoxynucleoside triphosphates under the conditions described. The maximum chain length was over 10,000 nt relative to a standard DNA ladder as judged by agarose gel electrophoresis. Hence, using the methods herein tails >5,000 bases long incorporating TTP and dUTP biotin are produced.

### EXAMPLE 3

### Labeling and Detection in Solution—Comparison of Hybridization Detection Efficiency of Single Biotin Labeled

Primer vs. Chain Extended Biotin Primer

Table 4 shows a comparison of the detectability of single biotin labeled probes to primers tailed to greater than 1 kb using biotin-dUTP in varying proportions to TTP. Tailing reactions were performed essentially as described above. The tailed oligomers and controls were then hybridized to a complementary lambda capture probe sequence attached to Costar plastic plates and washed in 3x in 1x SSC and excess fluid aspirated. The plates were then incubated in Streptavidin-HRP (1 ug/ml in binding buffer) for 15 minutes at 37° C., washed extensively and bound HRP detected with tetramethyl-benzidine for 1 hour at 37° C. The signal from 5.4×10⁷ molecules of singly labeled lambda RC biotin was 0.196.

TABLE 4
Comparison of Hybridization Detection Efficiency
of Single Biotin labeled
Lambada RC Primer vs. Long Tailed Lambda RC Biotin Primer

| | Estimated # | Length | Signal |
|---|---|---|---|
| Probe | Biotin/molecule | (nt) | Enhancement |
| Control-no primer | 0 | | |
| Lambda RC Biotin | 1 | 20 | 1 |
| Lambada RC Biotin tailed | 2 | ˜900 | 8.2 |
| Lambda RC Biotin tailed | 50 | ˜2000 | 98 |
| Lambda RC Biotin tailed | 75 | ˜3000 | 98 |
| Lambda RC Biotin tailed | 1000 | >10,000 | 9200 |

### EXAMPLE 4

Solid Phase Labeling—Determination of Chain Growth Efficiency on Hybridized DNA Probes

To demonstrate this effect on solid phase bound primers, DNA capture probes attached to plastic plates were prepared. The capture probes represent sequences complementary to the DNA probes described in Example 1. Capture probes attached to the plastic plates were then capped using ddATP as described above. The DNA probes were then hybridized to immobilized capture sequences on plastic plates, washed 3× in 1× SSC and excess fluid aspirated. Tailing reactions were then performed as described above in a total volume of 100 ul. At the completion of the tailing reaction, each well of the plate wash was washed extensively with 1× SSC wash buffer.

The tailed oligomers were then removed using ammonium hydroxide to denature the hybrids and elute them from the surface for characterization. Efficient tailing of the probes could then be observed, rendering the probes detectable.

### EXAMPLE 5

Solid Phase Labeling—Tailing on Probes Bound to Streptavidin-Agarose Beads Using Fluorescein-DUTP and Chain Release with RNase A

In this example, biotinylated oligonucleotides with the sequence biotin TTTTTTTTTTTTTTTTTTrU (SEQ ID No. 1; 3 separate lots) were bound to Streptavidin-agarose beads (Sigma, St. Louis, Mo.) and washed in binding buffer (0.1M Tris, pH 7.5, 0.1M NaCI) until no further material appeared in the wash solution. Tailing reactions were then performed as described above in a total volume of 50 ul containing TTP and fluorescein-dUTP (10:1) and incubated overnight at 37° C. At the completion of the tailing reaction, each well of the plate was washed extensively with 1× SSC wash buffer. The bound tailed oligomers were then released by treatment with 25ug/ml RNase A for 45 minutes at room temperature and the products analyzed on polyacrylamide gels.

The results, in which the size of the RNase released chains were compared to untreated controls and a series of other tailed oligomer primers, demonstrated that solid phase bound oligonucleotide primers terminated in rU were effectively chain extended by terminal transferase in the presence of fluorescein-dUTP and were subsequently specifically released by RNase A treatment.

### EXAMPLE 6

Detection of Chain Growth Directly on Solid Surfaces Using Fluorescein-dUTP Incorporation

The incorporation of dye-labeled nucleotide triphosphates onto primers hybridized to complementary capture probes bound to plastic surfaces demonstrate the feasibility of constructing polymer tails containing multiple fluorescent dyes. After construction of the plates, capping any reactive capture probes and hybridization of the detection probe is performed as described above. The detection probe is tailed using terminal transferase and fluorescein-dUTP/TTP (1:10) under conditions described in Example 2. The plates containing the tailed bound detection probe are washed extensively with 1× SSC (5×300 ul at room temperature), and then treated with 100 ul wash buffer and observed under UV light. Fluorescent signal is observed only in wells containing the detection probe. Best results are obtained when the primer sequence contains a non-hybridizable DNA tail of at least 3 bases.

### EXAMPLE 7

Detection of Chain Growth Directly on Solid Surfaces Using Biotin-dUTP Incorporation

The incorporation of biotin-dUTP labeled nucleotide triphosphates onto hybridized primers demonstrated the construction of polymer tails containing multiple biotin labels. After construction of the plates, capping any reactive capture probes and hybridization of the detection probe as described in Example 4 above, the detection probe was tailed using terminal transferase and biotin-dUTP/TTP (1:10) under conditions described in Example 2. The plates containing the tailed bound detection probe were washed extensively with 1× SSC (5×300 ul at room temperature), and then treated with 100 ul Streptavidin-HRP conjugate (1 ug/ml in 1× SSC). After 30 minutes incubation at 37° C., unbound Streptavidin-HRP conjugate was removed by washing several times in 1× SSC. Plates were then developed with 100 ul tetramethyl benzidine substrate for 100 minutes at 37° C. and the resulting color measured in a microplate reader.

The results demonstrated good biotin incorporation into primers and significant background reduction in the capped control wells. Best results were obtained when the primer sequence contained a non-hybridizable DNA tail of at least 3 bases.

As noted, all publications, patents and patent applications cited herein are incorporated herein by references as if each such publication, patent, or patent application was specifically and individually indicated to be incorporated herein by reference and was included in its entirety.

Since modifications will be apparent to those of skill in the art, it is intended that this invention be limited only by the scope of the appended claims.

1. A method, comprising:
a) treating nucleic acid molecules or modified nucleic acids in a sample with a reagent or reagents that render the nucleic acid chains unextendable by a non-template-dependent enzyme; and
b) hybridizing the treated molecules with a nucleic acid probe that includes an extendable terminus, under conditions whereby hybrids form; and
c) treating any hybrids formed with an non-template dependent chain elongating enzyme and substrates therefor, whereby any hybridized probe is extended.

2. The method of claim 1, wherein in step c) the non-template dependent chain elongating enzyme is a telomerase.

3. The method of claim 1, wherein the substrates comprise detectable moieties.

4. A method of detecting a nucleic acid probe added to a sample containing nucleic acids comprising the steps of:
(a) treating the sample with a chain terminating reagent to prevent polynucleotide chain growth from the nucleic acid in the sample;
(b) contacting the sample with the probe containing a terminus capable of elongation by a chain extending enzyme, wherein said probe hybridizes to the nucleic acid in the sample;
(c) contacting the sample with a chain extending enzyme and its substrates, thereby elongating the probe; and
(d) detecting the elongated hybridized probe.

5. The method of claim 4, where in the chain terminating reagent reacts directly with the sample to prevent polynucleotide growth.

6. The method in claim 4, wherein the chain terminating reagent is an enzyme substrate that in the presence of the enzyme reacts directly with the sample to prevent polynucleotide growth.

7. The method of claim 6, wherein the enzyme substrate is a nucleotide lacking a reactive hydroxyl.

8. The method of claim 6, wherein the enzyme substrate is a dideoxynucleotide.

9. The method of claim 4, wherein the chain extending enzyme is a telomerase.

10. The method of claim 4, where in the telomerase is terminal deoxynucleotidyl transferase.

11. The method of claim 4, wherein the chain extending enzyme is a polymerase.

12. The method of claim 4, wherein the chain extending enzyme is a polynucleotide phosphorylase.

13. The method of claim 4, wherein the substrates comprise nucleoside triphosphates labeled with fluorescent moieties.

14. The method of claim 13, wherein the substrate comprises a nucleoside triphosphate labeled with fluorescein dUTP.

15. The method of claim 13, wherein the substrate comprises a nucleoside triphosphate labeled with fluorescein dCTP.

16. The method of claim 4, wherein the substrate is a nucleoside triphosphate comprising a reporter group.

17. The method of claim 4, wherein the substrate is a nucleoside labeled with biotin dUTP.

18. The method of claim 4, wherein the substrate is a nucleoside labeled with digoxigenin dUTP.
